(19) United States
(12) Patent Application Publication
(10) Pub. No.: US 2024/0415896 A1
(43) Pub. Date: Dec. 19, 2024

(54) ANTIMICROBIAL COMPOSITIONS

(71) Applicant: Cornell University, Ithaca, NY (US)

(72) Inventors: Kenneth Simpson, Ithaca, NY (US); Shlying Zhang, Ithaca, NY (US)

(21) Appl. No.: 18/700,838

(22) PCT Filed: Oct. 13, 2022
(86) PCT No.: PCT/US2022/046530

§ 371 Date: Apr. 12, 2024

Related U.S. Application Data
(60) Provisional application No. 63/255,259, filed on Oct. 13, 2021.

(51) Int. Cl. A61K 35/74; A23L 33/135; A61K 45/06; A61P 31/04; C12N 1/20; C12R 1/01

(52) U.S. Cl. CPC A61K 35/74; A23L 33/135; A61K 45/06; A61P 31/04; C12N 1/20; C12R 2001/01

(57) ABSTRACT

The present invention relates to antimicrobial compounds isolated from Faecalibacterium prausnitzii supernatants, composition containing the compounds, and their use to inhibit bacterial growth or prevent bacterial infection, inhibit the virulence of N bacteria such as E. coli, and to treat diseases such as irritable bowel disease, Crohn's disease, and ulcerative colitis.

### CROSS REFERENCE TO RELATED APPLICATION

This application claims priority to and the benefit of U.S. Provisional Application No. 63/255,259, filed Oct. 13, 2021, which is hereby incorporated by reference in its entirety.

### FIELD OF INVENTION

The present invention relates to antimicrobial compounds isolated from Faecalibacterium prausnitzii supernatants, composition containing the compounds, and their use to inhibit bacterial growth or prevent bacterial infection, inhibit the virulence of bacteria such as E. coli, and to treat diseases such as irritable bowel disease, Crohn's disease, and ulcerative colitis.

### BACKGROUND OF THE INVENTION

Gut microbiota is known to have a role in shaping key aspects of postnatal life, such as the development of the immune system (Mazmanian et al., (2005) Cell 122(1): 107-118; Peterson et al., (2007) Cell Host Microbe 2(5): 328-339), and influencing the host's physiology, including energy balance. Transplanting the gut microbiota from normal mice into germ-free recipients increased their body fat without any increase in food consumption, raising the possibility that the composition of the microbial community in the gut affects the amount of energy extracted from the diet (Backhed et al., (2004) Proc Natl Acad Sci USA 101(44): 15718-15723). There is at least one type of obesity-associated gut microbiome characterised by higher relative abundance of Firmicutes or a higher Firmicutes to Bacteroidetes ratio (Ley et al., (2005) Proc Natl Acad Sci USA 102(31): 11070-11075; Tumbaugh et al., (2006) Nature 444(7122): 1027-1031). The role of intestinal microbiota in disease has also been shown. Gut microbes serve their host by functioning as a key interface with the environment; for example, they can protect the host organism from pathogens that cause infectious diarrhea. A decreased diversity of fecal microbiota and specifically a reduced diversity of Firmicutes in Crohn disease patients has been reported (Manichanh et al., (2006) Gut 55(2): 205-211), while it was recently shown that Faecalibacterium prausnitzii displays anti-inflammatory action and can potentially be used for the treatment of this disease (Sokol et al., (2008) Proc Natl Acad Sci USA 105(43): 16731-16736).

### SUMMARY OF THE INVENTION

The present invention relates to antimicrobial compounds isolated from Faecalibacterium prausnitzii supernatants, composition containing the compounds, and their use to inhibit bacterial growth or prevent bacterial infection, inhibit the virulence of bacteria such as E. coli, and to treat diseases such as acute gastroenteritis, traveler's diarrhea, irritable bowel syndrome, and inflammatory bowel diseases including Crohn's disease and ulcerative colitis.

In some preferred embodiments, the present invention provides compositions comprising a heat-stable secreted compound(s) from F. prausnitzii, the compound(s) characterized in having a molecular weight of less than 10 kDa.

In some alternative preferred embodiments, the present invention provides compositions comprising a F. prausnitzii supernatant fraction characterized in comprising heat-stable compounds having a molecular weight of less than 10 kDa and being substantially free of compounds of greater than 10 kDa in weight.

In other alternative embodiments, the present invention provides composition comprising heat-stable secreted compound(s) from F. prausnitzii.

In some preferred embodiments, the heat-stable secreted compound(s) are characterized in having a molecular weight of less than 8 kDa. In some preferred embodiments, the heat-stable secreted compound(s) are characterized in having a molecular weight of less than 6 kDa. In some preferred embodiments, the heat-stable secreted compound(s) are characterized in having a molecular weight of less than 5 kDa. In some preferred embodiments, the heat-stable secreted compound(s) are characterized in having a molecular weight of less than 3 kDa.

In some preferred embodiments, the compositions comprise heat-stable secreted compound(s) from a single F. prausnitzii strain. In some preferred embodiments, the compositions comprise heat-stable secreted compound(s) from a two or more F. prausnitzii strains. In some preferred embodiments, the compositions comprise heat-stable secreted compound(s) from one to five F. prausnitzii strains. In some preferred embodiments, the compositions comprise heat-stable secreted compound(s) from one to three F. prausnitzii strains.

In some preferred embodiments, the heat-stable compounds are from F. prausnitzii strain FP266. In some preferred embodiments, the heat-stable compounds are from F. prausnitzii strain FP267. In some preferred embodiments, the heat-stable compounds are from F. prausnitzii strain DSM17677.

In some preferred embodiments, the heat-stable compounds are obtained by culturing F. prausnitzii to provide a culture supernatant and heating the culture supernatant under conditions sufficient to denature proteins in the supernatant. In some preferred embodiments, the conditions sufficient to denature proteins in the supernatant comprise heating the supernatant to 100 degrees Celsius for 3 minutes. In some preferred embodiments, the culture supernatant is filtered or dialyzed to provide a fraction comprising molecules of less than 10 kDa.

In some preferred embodiments, the compositions further comprise a pharmaceutically acceptable excipient or carrier. In some preferred embodiments, the pharmaceutically acceptable excipient or carrier is not naturally associated with the F. prausnitzii heat-stable secreted compound(s).

In some preferred embodiments, the compositions are formulated as a powder, bolus, gel, liquid drench, capsule, tablet, emulsion, syrup, gummi or paste. In some preferred embodiments, the formulations comprise an effective amount of the compositions comprising a heat-stable secreted compound(s) from F. prausnitzii described above. In some preferred embodiments, the effective amount that reduces or modulates the growth and/or virulence of a pathogenic bacteria, for example E. coli, Klebsiella, Staphylococcus aureus, Salmonella spp., Listeria spp., or F. nucleatum.

In some preferred embodiments, the compositions comprise at least a second active agent. In some preferred embodiments, the at least a second active agent is selected from the group consisting of a prebiotic agent, a probiotic agent, and anti-inflammatory agent and an anti-bacterial agent and combinations thereof.

In some preferred embodiments, the present invention provides methods of inhibiting or reducing bacterial growth in a subject in need thereof comprising administering any one of the compositions described above to the subject. In some preferred embodiments, any one of the compositions described above is provided for use in inhibiting bacterial growth, treating a bacterial infection, or preventing a bacterial infection in a subject in need thereof. In some preferred embodiments, the bacteria is selected from the group consisting of E. coli, Klebsiella, Staphylococcus aureus, Salmonella spp., Listeria spp., and F. nucleatum. In some preferred embodiments, the E. coli is a Multiple Drug Resistant (MDR) E. coli strain. In some preferred embodiments, the E. coli is an adherent invasive E. coli (AIEC) strain. In some preferred embodiments, the adherence and/or invasiveness of the AIEC strain is reduced or inhibited. In some preferred embodiments, the E. coli is a cancer-associated E. coli strain. In some preferred embodiments, expression of the polyketide synthase gene (pks) is reduced or inhibited.

In some preferred embodiments, the present invention provides methods of reducing the virulence of a bacteria in a subject in need thereof comprising administering any one of the compositions described above to the subject. In some preferred embodiments, any one of the compositions described above is provided for use in inhibiting, treating or preventing virulence of a bacteria in a subject in need thereof. In some preferred embodiments, the bacteria is selected from the group consisting of E. coli, Klebsiella, Staphylococcus aureus, Salmonella spp., Listeria spp., and F. nucleatum. In some preferred embodiments, the E. coli is a Multiple Drug Resistant (MDR) E. coli strain. In some preferred embodiments, the E. coli is an adherent invasive E. coli (AIEC) strain. In some preferred embodiments, the adherence and/or invasiveness of the AIEC strain is reduced or inhibited. In some preferred embodiments, the E. coli is a cancer-associated E. coli strain. In some preferred embodiments, expression of the polyketide synthase gene (pks) is reduced or inhibited.

In some preferred embodiments, the present invention provides methods of treating, inhibiting or preventing acute gastroenteritis, traveler's diarrhea, irritable bowel syndrome, Crohn's disease or ulcerative colitis in a subject in need thereof comprising administering a any one of the compositions described above to the subject. In some preferred embodiments, the present invention provides any one of the compositions described above for use to treat, prevent or inhibit acute gastroenteritis, traveler's diarrhea, irritable bowel syndrome, Crohn's disease or ulcerative colitis in a subject in need thereof.

In some preferred embodiments, the present invention provides methods of inhibiting expression of the polyketide synthase gene (pks) gene by cancer-associated E. coli strains in a subject in need thereof comprising administering to the subject any one of the compositions described above. In some preferred embodiments, the present invention provides any one of the compositions described above for use to prevent or inhibit expression of the polyketide synthase gene (pks) gene by cancer-associated E. coli strains in a subject in need thereof.

In some preferred embodiments, the present invention provides methods of preventing or inhibiting adherence and/or invasiveness by AIEC E. coli strains in a subject in need thereof comprising administering to the subject any one of the compositions described above. In some preferred embodiments, the present invention provides any one of the compositions described above for use to prevent or inhibit adherence and/or invasiveness by AIEC E. coli strains in a subject in need thereof.

In some preferred embodiments, the present invention provides a food product comprising any one of the compositions described above in combination with one or more food ingredients selected from the group consisting of a fat, a carbohydrate and a protein. In some preferred embodiments, the one or more food ingredients does not naturally occur in F. prausnitzii. In some preferred embodiments, the present invention provides wherein the one or more food ingredient is obtained from a source other than F. prausnitzii. In some preferred embodiments, the food product is a solid food. In some preferred embodiments, the food product is a semi-solid food. In some preferred embodiments, the food product is a beverage. In some preferred embodiments, the food product is a baked good. In some preferred embodiments, the food product is a food bar.

### DESCRIPTION OF THE FIGURES

FIG. 1 provides a graph demonstrating the effect of F. prausnitzii supernatants from F. prausnitzii strains DSM (DSM17677), 30, 24, 266 and 267 on E. coli 541-1 growth.

FIGS. 2A and 2B provide graphs demonstrating the effect of F. prausnitzii supernatants (F. prausnitzii strains 266 and 30) compared to culture media (VTR2RF, M9) on growth of Salmonella spp. See FIGS. 2A and 2B.

FIGS. 3A and 3B provides graphs of data demonstrating that the growth inhibition observed in FIGS. 1 and 2 against E. coli strain 541-1 is not due to nutrient depletion.

FIGS. 4A and 4B provide data demonstrating the effect of dialysis (6-8 kDa) and boiling on growth inhibition.

FIG. 5 provides a graph demonstrating the effect of F. prausnitzii 266 supernatant on E. coli LF82 virulence gene expression.

FIGS. 6A and 6B provides the results from Sephedex G25 filtration of selected supernatants on ability to inhibit growth of AIEC E. coli 541-1.

FIGS. 7A, 7B and 7C provide demonstrating that heat stable filterable secreted products of F. prausnitzii 266 inhibit the growth of Salmonella (7A), Klebsiella (7B) and Staphylococcus spp. (7C).

FIG. 8 is a graph of data demonstrating that the predominant antibacterial effect of F. prausnitzii secreted products is mediated by constituents <3 kDa.

FIG. 9 provides a graph showing the heat stability of a 3K filtrate of F. prausnitzii 267 supernatant.

FIGS. 10A and 10B provides graphs showing that filtered and heat treated F. prausnitzii 3K filtrate inhibits the growth of multiple drug resistant (MDR) E. coli strains.

FIGS. 11A, 11B and 11C provide graphs showing that filtered and heat treated F. prausnitzii 3K filtrate inhibits the growth of E. coli strains that are associated with the development of colon cancer (E. coli HM164 (FIG. TTA), HM213 (FIG. 11B) and HM334 (FIG. 11C)).

FIG. 12 is a graph showing that treated F. prausnitzii 3K filtrate inhibits the growth of F. nucleatum.

FIGS. 13A, 13B, 13C, 13D, 13E and 13F provide graphs showing that the DSM17677 filtrate inhibits the growth of AIEC (Adherent Invasive E. coli) E. coli strains (e.g., strains 541-1 (FIG. 13A), 552-2 (FIG. 13B) and LF82 (FIG. 13C)), Salmonella typhi (FIG. 13D), Listeria (FIG. 13E) and S. aureus (FIG. 13F).

FIGS. 14A and 14B provides graphs showing that F. prausnitzii secreted products reduce the ability of AIEC to adhere to (FIG. 14A) and invade (FIG. 14B) intestinal epithelial cells (Caco2) in vitro.

FIGS. 15A, 15B and 15C provides graphs showing that F. prausnitzii strain FP266 (FIG. 15A), FP267 (FIG. 15B) and DSM (FIG. 15C) secreted products inhibit transcription of the pks gene in cancer-associated E. coli.

### DEFINITIONS

To facilitate an understanding of the present invention, a number of terms and phrases are defined below: As used herein, the terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are gram negative or gram positive. “Gram negative” and “gram positive” refer to staining patterns with the Gram-staining process that is well known in the art. (See e.g., Finegold and Martin, Diagnostic Microbiology, 6th Ed., CV Mosby St. Louis, pp. 13-15 [1982]). “Gram positive bacteria” are bacteria that retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red.

As used herein, the term “heat-stable” when in reference to compounds in an F. prausnitzii supernatant refers to compounds that retain one or more activities (e.g., inhibition of growth and/or virulence of bacteria such as Escherichia coli (including Multiple Drug Resistant (MDR) E. coli strains, Adherent Invasive E. coli (AIEC) strains, and cancer-associated E. coli strains), Salmonella spp., Klebsiella, Staphylococcus spp., Listeria spp., and Fusobacterium nucleatum) following heating at 100 degrees Celsius for 3 minutes.

As used herein, the term “cancer-associated E. coli” refers to E. coli strains that comprise and express polyketide synthase (pks) island genes which are associated with an increased risk of colon cancer.

As used herein, the term “in vitro” refers to an artificial environment and to processes or reactions that occur within an artificial environment. In vitro environments can consist of, but are not limited to, test tubes, microtiter plates, and the like. The term “in vivo” refers to the natural environment (e.g., an animal or a cell) and to processes or reactions that occur within a natural environment.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “substantially free” when used in reference to the content of compounds of stated molecular weight ranges in an F. prausnitzii supernatant means that the composition is comprised of compounds below the cut off value for a suitable molecular weight cut-off filter or dialysis system.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

As used herein, a “subject” is an animal, preferably a mammal such as a human, domestic animal, or companion animal or other vertebrate. Mammals are understood to include, but are not limited to, murines, simians, humans, bovines, cervids, equines, porcines, canines, felines, etc.

As used herein, an “effective amount” is an amount sufficient to effect beneficial or desired results. An effective amount can be administered in one or more administrations,

As used herein, “co-administration” refers to administration of more than one agent or therapy to a subject. Co-administration may be concurrent or, alternatively, the chemical compounds described herein may be administered in advance of or following the administration of the other agent(s). One skilled in the art can readily determine the appropriate dosage for co-administration. When co-administered with another therapeutic agent, both the agents may be used at lower dosages. Thus, co-administration is especially desirable where the claimed compounds are used to lower the requisite dosage of known toxic agents.

As used herein, the term “toxic” refers to any detrimental or harmful effects on a cell or tissue.

As used herein, a “pharmaceutical composition” is intended to include the combination of an active agent with a carrier, inert or active, making the composition suitable for diagnostic or therapeutic use in vivo, in vivo or ex vivo.

As used herein, the term “pharmaceutically acceptable carrier” encompasses any of the standard pharmaceutical carriers, such as a phosphate buffered saline solution, water, and an emulsion, such as an oil/water or water/oil emulsion, and various types of wetting agents. The compositions also can include stabilizers and preservatives. For examples of carriers, stabilizers and adjuvants see Martin, Remington's Pharmaceutical Sciences, 15th Ed., Mack Publ. Co., Easton, PA (1975).

As used herein, the term “nutraceutical,” refers to a food substance or part of a food, which includes a probiotic bacterium. Nutraceuticals can provide medical or health benefits, including the prevention, treatment, or cure of a disorder.

### DETAILED DESCRIPTION OF THE INVENTION

The present invention relates to antimicrobial compounds isolated from Faecalibacterium prausnitzii supernatants, composition containing the compounds, and their use to inhibit bacterial growth or prevent bacterial infection, inhibit the virulence of bacteria such as Escherichia coli (including Multiple Drug Resistant (MDR) E. coli strains, Adherent Invasive E. coli (AIEC) strains, and cancer-associated E. coli strains), Salmonella spp., Klebsiella, Staphylococcus spp., Listeria spp., and Fusobacterium nucleatum and to treat or provide prophylaxis for diseases such as acute gastroenteritis, irritable bowel syndrome, and inflammatory bowel diseases including Crohn's disease and ulcerative colitis. In other embodiments, the compositions are useful for decreasing or inhibiting expression of E. coli genes associated with cancer, such as one or genes in the pks island.

Accordingly, in some preferred embodiments, the present invention provides compositions comprising a heat-stable secreted compound(s) from F. prausnitzii, the compound(s) are characterized in having molecular weight of less than 10 kDa. In some preferred embodiments, the compound(s) are characterized in having molecular weight of less than 8 kDa. In some preferred embodiments, the compound(s) are characterized in having molecular weight of less than 6 kDa. In some preferred embodiments, the compound(s) are characterized in having molecular weight of less than 5 kDa. In some preferred embodiments, the compound(s) are characterized in having molecular weight of less than 3 kDa.

In some further preferred embodiments, the present invention provides a composition comprising a F. prausnitzii supernatant fraction characterized in comprising heat-stable compounds having a molecular weight of less than 10 kDa and being substantially free of compounds of greater than 10 kDa in weight. In some preferred embodiments, the F. prausnitzii supernatant fraction characterized in comprising heat-stable compounds having a molecular weight of less than 8 kDa and being substantially free of compounds of greater than 8 kDa in weight. In some preferred embodiments, the F. prausnitzii supernatant fraction characterized in comprising heat-stable compounds having a molecular weight of less than 6 kDa and being substantially free of compounds of greater than 6 kDa in weight. In some preferred embodiments, the F. prausnitzii supernatant fraction characterized in comprising heat-stable compounds having a molecular weight of less than 5 kDa and being substantially free of compounds of greater than 5 kDa in weight. In some preferred embodiments, the F. prausnitzii supernatant fraction characterized in comprising heat-stable compounds having a molecular weight of less than 3 kDa and being substantially free of compounds of greater than 3 kDa in weight.

In some further preferred embodiments, the present invention provides a composition comprising a F. prausnitzii supernatant fraction characterized in comprising heat-stable compounds having a molecular weight of less than 10 kDa and being further characterized in comprising less than 5.0% by dry weight of compounds of greater than 10 kDa. In some further preferred embodiments, the present invention provides a composition comprising a F. prausnitzii supernatant fraction characterized in comprising heat-stable compounds having a molecular weight of less than 10 kDa and being further characterized in comprising less than 1.0% by dry weight of compounds of greater than 10 kDa. In some further preferred embodiments, the present invention provides a composition comprising a F. prausnitzii supernatant fraction characterized in comprising heat-stable compounds having a molecular weight of less than 8 kDa and being further characterized in comprising less than 5.0% by dry weight of compounds of greater than 8 kDa. In some further preferred embodiments, the present invention provides a composition comprising a F. prausnitzii supernatant fraction characterized in comprising heat-stable compounds having a molecular weight of less than 8 kDa and being further characterized in comprising less than 1.0% by dry weight of compounds of greater than 8 kDa. In some further preferred embodiments, the present invention provides a composition comprising a F. prausnitzii supernatant fraction characterized in comprising heat-stable compounds having a molecular weight of less than 6 kDa and being further characterized in comprising less than 5.0% by dry weight of compounds of greater than 6 kDa. In some further preferred embodiments, the present invention provides a composition comprising a F. prausnitzii supernatant fraction characterized in comprising heat-stable compounds having a molecular weight of less than 6 kDa and being further characterized in comprising less than 1.0% by dry weight of compounds of greater than 6 kDa. In some further preferred embodiments, the present invention provides a composition comprising a F. prausnitzii supernatant fraction characterized in comprising heat-stable compounds having a molecular weight of less than 5 kDa and being further characterized in comprising less than 5.0% by dry weight of compounds of greater than 5 kDa. In some further preferred embodiments, the present invention provides a composition comprising a F. prausnitzii supernatant fraction characterized in comprising heat-stable compounds having a molecular weight of less than 5 kDa and being further characterized in comprising less than 1.0% by dry weight of compounds of greater than 5 kDa. In some further preferred embodiments, the present invention provides a composition comprising a F. prausnitzii supernatant fraction characterized in comprising heat-stable compounds having a molecular weight of less than 3 kDa and being further characterized in comprising less than 5.0% by dry weight of compounds of greater than 3 kDa. In some further preferred embodiments, the present invention provides a composition comprising a F. prausnitzii supernatant fraction characterized in comprising heat-stable compounds having a molecular weight of less than 3 kDa and being further characterized in comprising less than 1.0% by dry weight of compounds of greater than 3 kDa.

In some preferred embodiments, the F. prausnitzii supernatant fraction characterized in comprising heat-stable compounds having a molecular weight of less than 8 kDa and being substantially free of compounds of greater than 8 kDa in weight. In some preferred embodiments, the F. prausnitzii supernatant fraction characterized in comprising heat-stable compounds having a molecular weight of less than 6 kDa and being substantially free of compounds of greater than 6 kDa in weight. In some preferred embodiments, the F. prausnitzii supernatant fraction characterized in comprising heat-stable compounds having a molecular weight of less than 5 kDa and being substantially free of compounds of greater than 5 kDa in weight. In some preferred embodiments, the F. prausnitzii supernatant fraction characterized in comprising heat-stable compounds having a molecular weight of less than 3 kDa and being substantially free of compounds of greater than 3 kDa in weight. In some preferred embodiments, the present invention provides compositions comprising secreted F. prausnitzii heat-stable compounds. In some preferred embodiments, the secreted F. prausnitzii heat-stable compounds are obtained by heating an F. prausnitzii culture supernatant to 100 degrees Celsius for three minutes.

In some preferred embodiments, the heat-stable compounds are obtained by culturing F. prausnitzii to provide a culture supernatant and processing the culture supernatant to provide a fraction comprising molecules of less than 10 kDa. In some preferred embodiments, the heat-stable compounds are obtained by culturing F. prausnitzii to provide a culture supernatant and processing the culture supernatant to provide a fraction comprising molecules of less than 8 kDa. In some preferred embodiments, the heat-stable compounds are obtained by culturing F. prausnitzii to provide a culture supernatant and processing the culture supernatant to provide a fraction comprising molecules of less than 6 kDa. In some preferred embodiments, the heat-stable compounds are obtained by culturing F. prausnitzii to provide a culture supernatant and processing the culture supernatant to provide a fraction comprising molecules of less than 5 kDa. In some preferred embodiments, the heat-stable compounds are obtained by culturing F. prausnitzii to provide a culture supernatant and processing the culture supernatant to provide a fraction comprising molecules of less than 3 kDa. In some embodiments, size exclusion filters, chromatography columns or dialysis systems are utilized to provide fractions containing molecules of the desired size. In each of the embodiments, the supernatant may optionally be heated before or after filtration, for example for 3 minutes at a temperature of 100 degrees Celsius.

The present invention is not limited to the use of compounds from any particular strain of F. prausnitzii. In some preferred embodiments, the strains are, for example, swine, bovine or human F. prausnitzii strains. In some preferred embodiments, the strain is the swine 266 or 277 strain. In some preferred embodiments, the strain is the bovine 24 or 30 strain. In some preferred embodiments, the strain is the human DSM strain (DSM17677). In other preferred embodiments, the F. prausnitzii used in the compositions and methods of the present invention is isolated as described in Foditsch et al., PLOS ONE DOI:10.1371/journal.pone.0116465(2014), incorporated herein by reference in its entirety. It will be understood to those of ordinary skill in the art that suitable strains of F. prausnitzii may be obtained as described in Foditsch (2014) and screened as described herein for antimicrobial activity or bacterial taming activity. Thus, the current invention is not limited to the specific strains described herein. Multiple strains with antimicrobial activity are described in the examples and additional strains for use in the methods and compositions if the present invention can be obtained as described herein.

In some preferred embodiments, the compositions comprise heat-stable compounds of the indicated molecular weight range from a single F. prausnitzii strain. In some preferred embodiments, the compositions comprise heat-stable compounds of the indicated molecular weight range from two or more F. prausnitzii strains. In some preferred embodiments, the compositions comprise heat-stable compounds of the indicated molecular weight range from 1 to 5 F. prausnitzii strains. In some preferred embodiments, the compositions comprise heat-stable compounds of the indicated molecular weight range from 1 to 3 F. prausnitzii strains.

In some embodiments, compositions and formulations of the present invention comprise an effective amount of the F. prausnitzii composition, for example, a F. prausnitzii culture supernatant with a defined molecular weight range. In some embodiments, the effective amount is an amount sufficient to inhibit the growth of a target microorganism. In some embodiments, the effective amount is an amount sufficient to prevent the growth of a target microorganism. In some embodiments, the amount is effective to reduce the amount of the microorganism by at least 90%, 80%, 70%, 60%, 50% or 25% in a subject being treated as compared to the amount of microorganism present prior to administration in a subject. In some embodiments, the amount is effective to reduce the growth of the amount of the microorganism by at least 90%, 80%, 70%, 60%, 50% or 25% in a subject being treated as compared to the amount of microorganism in the absence of treatment of a subject. The amount of the microorganism present can be determined, for example, by culturing a sample or swab taken from a subject that is being treated. In some embodiments, the amount is effective to treat or prevent an infection by the target microorganism. In some embodiments, the amount is effective to inhibit or modulate virulence (e.g., adhesion and/or invasiveness) of a target microorganism. In some embodiments, the amount is effective to decrease, inhibit, or modulate expression of one or more pks island genes (e.g., polyketide synthase (PKS), nonribosomal peptide synthetase (NRPS), and/or hybrid NRPS-PKS.) In some embodiments, the amount is effective to decrease, inhibit, or modulate production of colibactin.

As described in more detail below, the composition of the present invention may be provided with one or more pharmaceutically acceptable carriers or excipients and in a variety of different formulations.

The present invention further provides methods of using the compositions described above.

In some preferred embodiments, the compositions are administered to a subject in need thereof to inhibit the growth of a bacterial target organism. In some preferred embodiments, the compositions are administered to a subject in need thereof to modulate the growth of a bacterial target organism. In some preferred embodiments, the compositions are administered to a subject in need thereof to inhibit infection of the subject with a bacterial target organism. In some preferred embodiments, the compositions are administered to a subject in need thereof to treat or prevent a bacterial infection. In some embodiments, the compositions are used for prophylaxis of bacterial infection. The present invention is not limited to treatment of any particular bacterial infection. In some preferred embodiments, the bacteria is selected from the group consisting of Escherichia coli (including MDR E. coli strains, AIEC strains, and cancer-associated E. coli strains), Salmonella spp., Klebsiella spp, Staphylococcus spp., Listeria spp., and Fusobacterium nucleatum In some preferred embodiments, the bacteria is an MDR E. coli strain. In some preferred embodiments, the bacteria is an AIEC strain. In some preferred embodiments, the bacteria is a cancer-associated E. coli strain. In some preferred embodiments, the bacteria is a Salmonella spp. In some preferred embodiments, the bacteria is a Klebsiella ssp. In some preferred embodiments, the bacteria is a Staphylococcus spp. In some preferred embodiments, the bacteria is a Listeria spp. In some preferred embodiments, the bacteria is Fusobacterium nucleatum.

In some preferred embodiments, the composition are administered to a subject in thereof to treat, prevent, inhibit, or modulate the virulence of a target bacterial organism. In some preferred embodiments, the bacteria is a pathogenic strain of E. coli. In some preferred embodiments, the pathogenic strain of E. coli an MDR E. coli strain. In some preferred embodiments, the pathogenic strain of E. coli an AIEC strain. In some preferred embodiments, the pathogenic strain of E. coli a cancer-associated E. coli strain.

The compositions described above further find use in treatment, prevention, or inhibition of a variety of diseases of the digestive tract.

In some embodiments, the compositions of the present invention are used to treat a subject suffering from acute gastroenteritis, traveler's diarrhea, irritable bowel disease, and inflammatory bowel disease such as ulcerative colitis or Crohn's disease. In some preferred embodiments, the disease is acute gastroenteritis. In some preferred embodiments, the disease is traveler's diarrhea. In some preferred embodiments, the disease is irritable bowel disease. In some preferred embodiments, the disease is inflammatory bowel disease. In some preferred embodiments, the disease is ulcerative colitis. In some preferred embodiments, the disease is Crohn's disease. In some preferred embodiments, an effective amount of the composition is administered to the subject. In some preferred embodiments, administration of the composition of the present invention results in improvement of or modulation of one or more symptoms associated with acute gastroenteritis, traveler's diarrhea and irritable bowel diseases such as ulcerative colitis or Crohn's disease.

In some embodiments, the compositions of the present invention are used to provide prophylaxis in a subject for a disease selected from colon cancer, acute gastroenteritis, traveler's diarrhea, irritable bowel disease, and inflammatory bowel disease such as ulcerative colitis or Crohn's disease. In some preferred embodiments, the disease is colon cancer. In some preferred embodiments, the disease is acute gastroenteritis. In some preferred embodiments, the disease is traveler's diarrhea. In some preferred embodiments, the disease is irritable bowel disease. In some preferred embodiments, the disease is inflammatory bowel disease. In some preferred embodiments, the disease is ulcerative colitis. In some preferred embodiments, the disease is Crohn's disease. In some preferred embodiments, an effective amount of the composition is administered to the subject to provide prophylaxis.

In some embodiments, compositions comprise one or more additional active agents (i.e., at least one or more second active agents where the first active agent is the F. prausnitzii composition described above) and/or components (e.g., including but not limited to, additional additive selected from the group consisting of an energy substrate, a mineral, a vitamin, or combinations thereof).

In some preferred embodiments, the at least one or more second active agent is a probiotic agent. In some embodiments, compositions comprise one or more (e.g., 2 or more, 5 or more, 10 or more, etc.) additional strains of bacteria or other microorganisms (e.g., probiotic microorganisms). Examples include, but are not limited to, Lactobacillus acidophilus, L. lactis, L. plantarum, L. casei, Bacillus subtilis, B. lichenformis, Enterococcus faecium, Bifidobacterium bifidum, B. longum, B. thermophilum, Propionibacterium jensenii, yeast, or combinations thereof. In some embodiments, multiple strains of the same bacteria are utilized in combination.

In some embodiments, probiotic bacteria are live cells or freeze-dried cells. Freeze-dried bacteria can be stored for several years with maintained viability. In certain applications, freeze-dried bacteria are sensitive to humidity. One way of protecting the bacterial cells is to store them in oil. The freeze dried bacterial cells can be mixed directly with a suitable oil, or alternately the bacterial cell solution can be mixed with an oil and freeze dried together, leaving the bacterial cells completely immersed in oil. Suitable oils may be edible oils such as olive oil, rapeseed oil which is prepared conventionally or cold-pressed, sunflower oil, soy oil, maize oil, cotton-seed oil, peanut oil, sesame oil, cereal germ oil such as wheat germ oil, grape kernel oil, palm oil and palm kernel oil, linseed oil. The viability of freeze-dried bacteria in oil is maintained for at least nine months. Optionally live cells can be added to one of the above oils and stored.

In some preferred embodiments, the at least one or more second active agent is a prebiotic agent. Suitable prebiotic agents include indigestible plant fiber materials including, but not limited to, brans, flours, gums, fiber extracts and other plant fiber-containing materials prepared from plants and includes whole plant materials and portions of plants that contain plant. Some examples include, but are not limited to, whole grains, gum arabic, inulin, wheat bran, oat bran, psyllium, raw or dry chicory root, raw or dry Jerusalem artichoke, raw or dry dandelion greens, raw or dry garlic, raw or dry onion, raw or dry leek, raw or dry asparagus, marine algae (e.g., spirulina, chlorella, nostoc, etc.), arabinogalactan, raw or dried mushrooms. In some preferred embodiments, the at least one or more second active agent is an anti-inflammatory agent. Suitable anti-inflammatory agents include, but are not limited to, non-steroidal anti-inflammatory drugs (NSAIDs) including diclofenac (also known as Voltaren, Abitren, Allvoran, Almiral, Alonpin, Anfenax, Artrites, Betaren, Blesin, Bolabomin, Cataflam, Clofec, Clofen, Cordralan, Curinflam, Diclomax, Diclosian, Dicsnal, Difenac, Ecofenac, Hizemin, Inflamac, Inflanac, Klotaren, Lidonin, Monoflam, Naboal, Oritaren, Remethan, Savismin, Silino, Staren, Tsudohmin, Voltarol, Voren, Voveran, and Vurdon), diflunisal (also known as Dolobid, Adomal, Diflonid, Diflunil, Dolisal, Dolobis, Dolocid, Donobid, Dopanone, Dorbid, Dugodol, Flovacil, Fluniget, Fluodonil, Flustar, Ilacen, Noaldol, Reuflos, and Unisal), etodolac (also known as Lodine), fenoprofen (also known as Nalfon, Fenoprex, Fenopron, Fepron, Nalgesic, and Progesic), flurbiprofen (also known as Ansaid and Ocuflur), ibuprofen (also known as Rufen, Motrin, Aches-N-Pain, Advil, Nuprin, Dolgesic, Genpril, Haltran, Ibifon, Ibren, Ibumed, Ibuprin, Ibupro-600, Ibuprohm, Ibu-Tab, Ibutex, Ifen, Medipren, Midol 200, Motrin-IB, Cramp End, Profen, Ro-Profen, Trendar, Alaxan, Brofen, Alfam, Brufen, Algofen, Brufort, Amersol, Bruzon, Andran, Buburone, Anflagen, Butacortelone, Apsifen, Deflem, Artofen, Dolgit, Artril, Dolocyl, Bloom, Donjust, Bluton, Easifon, Ebufac, Emflam, Emodin, Fenbid, Fenspan, Focus, Ibosure, Ibufen, Ibufug, Ibugen, Ibumetin, Ibupirac, Imbun, Inabrin, Inflam, Irfen, Librofen, Limidon, Lopane, Mynosedin, Napacetin, Nobafon, Nobgen, Novogent, Novoprofen, Nurofen, Optifen, Paduden, Paxofen, Perofen, Proartinal, Prontalgin, Q-Profen, Relcofen, Remofen, Roidenin, Seclodin, Tarein, and Zofen), indomethacin (also known as Indameth, Indocin, Amuno, Antalgin, Areumatin, Argilex, Artherexin, Arthrexin, Artrinovo, Bavilon, Bonidon, Boutycin, Chrono-Indocid, Cidalgon, Confortid, Confortind, Domecid, Durametacin, Elemetacin, Idicin, Imbrilon, Inacid, Indacin, Indecin, Indocap, Indocen, Indocid, Indoflex, Indolag, Indolar, Indomed, Indomee, Indometacinum, Indometicina, Indometin, Indovis, Indox, Indozu, Indrenin, Indylon, Inflazon, Inpan, Lauzit, Liometace, Metacen, Metindon, Metocid, Mezolin, Mobilan, Novomethacin, Peralgon, Reflox, Rheumacid, Rheumacin, Salinac, Servindomet, Toshisan, and Vonum), ketoprofen (also known as Orudis, Alrheumat, Alrheumun, Alrhumat, Aneol, Arcental, Dexal, Epatec, Fastum, Keduril, Kefenid, Keprofen, Ketofen, Ketonal, Ketosolan, Kevadon, Mero, Naxal, Oruvail, Profenid, Salient, Tofen, and Treosin), ketorolac (also known as Toradol), meclofenamate (also known as Meclofen, Meclomen, and Movens), mefenamic acid (also known as Ponstel, Alpain, Aprostal, Benostan, Bonabol, Coslan, Dysman, Dyspen, Ecopan, Lysalgo, Manic, Mefac, Mefic, Mefix, Parkemed, Pondex, Ponsfen, Ponstan, Ponstyl, Pontal, Ralgec, and Youfenam), nabumetone (also known as Relafen), naproxen (also known as Naprosyn, Anaprox, Aleve, Apranax, Apronax, Arthrisil, Artrixen, Artroxen, Bonyl, Congex, Danaprox, Diocodal, Dysmenalgit, Femex, Flanax, Flexipen, Floginax, Gibixen, Headlon, Laraflex, Laser, Leniartil, Nafasol, Naixan, Nalyxan, Napoton, Napren, Naprelan, Naprium, Naprius, Naprontag, Naprux, Napxen, Narma, Naxen, Naxid, Novonaprox, Nycopren, Patxen, Prexan, Prodexin, Rahsen, Roxen, Saritilron, Sinartrin, Sinton, Sutony, Synflex, Tohexen, Veradol, Vinsen, and Xenar), oxaprozin (also known as Daypro), piroxicam (also known as Feldene, Algidol, Antiflog, Arpyrox, Atidem, Bestocam, Butacinon, Desinflam, Dixonal, Doblexan, Dolonex, Feline, Felrox, Fuldin, Indene, Infeld, Inflamene, Lampoflex, Larapam, Medoptil, Novopirocam, Osteral, Pilox, Piraldene, Piram, Pirax, Piricam, Pirocam, Pirocaps, Piroxan, Piroxedol, Piroxim, Piton, Posidene, Pyroxy, Reucam, Rexicam, Riacen, Rosic, Sinalgico, Sotilen, Stopen, and Zunden), sulindac (also known as Clinoril, Aflodac, Algocetil, Antribid, Arthridex, Arthrocine, Biflace, Citireuma, Clisundac, Imbaral, Lindak, Lyndak, Mobilin, Reumofil, Sudac, Sulene, Sulic, Sulindal, Suloril, and Sulreuma), tolmetin (also known as Tolectin, Donison, Midocil, Reutol, and Safitex), celecoxib (also known as Celebrex), meloxicam (also known as Mobic), rofecoxib (also known as Vioxx), valdecoxib (also known as Bextra), aspirin (also known as Anacin, Ascriptin, Bayer, Bufferin, Ecotrin, and Excedrin) and steroidal anti-inflammatory drugs including cortisone, prednisone and dexamethasone.

In some preferred embodiments, the at least one of more second active agent is an anti-bacterial agent. Suitable anti-bacterial agents include, but are not limited to, loracarbef, cephalexin, cefadroxil, cefixime, ceftibuten, cefprozil, cefpodoxime, cephradine, cefuroxime, cefaclor, neomycin/polymyxin/bacitracin, dicloxacillin, nitrofurantoin, nitrofurantoin macrocrystal, nitrofurantoin/nitrofuran mac, dirithromycin, gemifloxacin, ampicillin, gatifloxacin, penicillin V potassium, ciprofloxacin, enoxacin, amoxicillin, amoxicillin/clavulanate potassium, clarithromycin, levofloxacin, moxifloxacin, azithromycin, sparfloxacin, cefdinir, ofloxacin, trovafloxacin, lomefloxacin, methenamine, erythromycin, norfloxacin, clindamycin/benzoyl peroxide, quinupristin/dalfopristin, doxycycline, amikacin sulfate, vancomycin, kanamycin, netilmicin, streptomycin, tobramycin sulfate, gentamicin sulfate, tetracyclines, framycetin, minocycline, nalidixic acid, demeclocycline, trimethoprim, miconazole, colistimethate, piperacillin sodium/tazobactam sodium, paromomycin, colistin/neomycin/hydrocortisone, amebicides, sulfisoxazole, pentamidine, sulfadiazine, clindamycin phosphate, metronidazole, oxacillin sodium, nafcillin sodium, vancomycin hydrochloride, clindamycin, cefotaxime sodium, co-trimoxazole, ticarcillin disodium, piperacillin sodium, ticarcillin disodium/clavulanate potassium, neomycin, daptomycin, cefazolin sodium, cefoxitin sodium, ceftizoxime sodium, penicillin G potassium and sodium, ceftriaxone sodium, ceftazidime, imipenem/cilastatin sodium, aztreonam, cinoxacin, erythromycin/sulfisoxazole, cefotetan disodium, ampicillin sodium/sulbactam sodium, cefoperazone sodium, cefamandole nafate, gentamicin, sulfisoxazole/phenazopyridine, tobramycin, lincomycin, clindamycin hydrochloride, lansoprazole/clarithromycin/amoxicillin, alatrofloxacin, linezolid, bismuth subsalicylate/metronidazole/tetracycline, erythromycin/benzoyl peroxide, mupirocin, fosfomycin, pentamidine isethionate, imipenem/cilastatin, troleandomycin, gatifloxacin, chloramphenicol, cycloserine, neomycin/polymyxin B/hydrocortisone, ertapenem, meropenem, cephalosporins, fluconazole, cefepime, sulfamethoxazole, sulfamethoxazole/trimethoprim, penicillins, rifampin/isoniazid, erythromycin estolate, erythromycin ethylsuccinate, erythromycin stearate, ampicillin trihydrate, ampicillin/probenecid, sulfasalazine, sulfanilamide, sodium sulfacetamide, dapsone, doxycycline hyclate, trimenthoprim/sulfa, methenamine mandelate, plasmodicides, pyrimethamine, hydroxychloroquine, chloroquine phosphate, trichomonocides, anthelmintics, atovaquone, defensins, cathelicidins, bacitracin, bacitracin/polymyxin b, gentamycin, neomycin/polymyxin/dexameth, neomycin sulf/dexameth, sulfacetamide/prednisolone, sulfacetamide/phenylephrine, tobramycin sulfate/dexameth, and bismuth tribromophenate.

In some embodiments, the compositions of the present invention may preferably be dried, for example by spray drying, vacuum drying, freeze-drying or lyophilization. The resulting powder may preferably be formulated as a powder, such as a dispersible powder, bolus, gel, liquid drench, capsule, or paste.

In some embodiments, the compositions are formulated as part of a milk replacer (e.g., for administration to a neonatal or young animal). In some embodiments, compositions comprise one or more probiotic bacteria as described herein in combination with a milk protein (e.g., caseins or whey proteins).

In some embodiments, compositions are added to nutraceuticals, food products, or foods. In some embodiments, to give the composition or nutraceutical a pleasant taste, flavoring substances such as for example mints, fruit juices, licorice, Stevia rebaudiana, steviosides or other calorie free sweeteners, rebaudioside A, essential oils like eucalyptus oil, or menthol can optionally be included in compositions of embodiments of the present invention.

In some embodiments, compositions are formulated in pharmaceutical compositions. The bacteria of embodiments of the invention may be administered alone or in combination with pharmaceutically acceptable carriers or diluents, and such administration may be carried out in single or multiple doses.

Compositions may, for example, be in the form of tablets, resolvable tablets, capsules, bolus, drench, pastes, pills sachets, vials, hard or soft capsules, aqueous or oily suspensions, aqueous or oily solutions, emulsions, powders, granules, syrups, elixirs, lozenges, reconstitutable powders, liquid preparations, creams, troches, hard candies, sprays, chewing-gums, creams, salves, jellies, gels, pastes, toothpastes, rinses, dental floss and tooth-picks, liquid aerosols, dry powder formulations, HFA aerosols or organic or inorganic acid addition salts.

The pharmaceutical compositions of embodiments of the invention may be in a form suitable for oral, topical, buccal administration. Depending upon the disorder and patient to be treated and the route of administration, the compositions may be administered at varying doses.

For oral or buccal administration, bacteria of embodiments of the present invention may be combined with various excipients. Solid pharmaceutical preparations for oral administration often include binding agents (for example syrups, acacia, gelatin, tragacanth, polyvinylpyrrolidone, sodium lauryl sulphate, pregelatinized maize starch, hydroxypropyl methylcellulose, starches, modified starches, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone and sodium alginate), disintegrants (such as starch and preferably corn, potato or tapioca starch, alginic acid and certain complex silicates, polyvinylpyrrolidone, gelatin, acacia, sodium starch glycollate, microcrystalline cellulose, crosscarmellose sodium, crospovidone, hydroxypropyl methylcellulose and hydroxypropyl cellulose), lubricating agents (such as magnesium stearate, sodium lauryl sulfate, talc, silica polyethylene glycol waxes, stearic acid, palmitic acid, calcium stearate, camuba wax, hydrogenated vegetable oils, mineral oils, polyethylene glycols and sodium stearyl fumarate) and fillers (including high molecular weight polyethylene glycols, lactose, calcium phosphate, glycine magnesium stearate, starch, rice flour, chalk, gelatin, microcrystalline cellulose, calcium sulphate, and lactitol). Such preparations may also include preservative agents and anti-oxidants.

Liquid compositions for oral administration may be in the form of, for example, emulsions, syrups, or elixirs, or may be presented as a dry product for reconstitution with water or other suitable vehicle before use. Such liquid compositions may contain conventional additives such as suspending agents (e.g. syrup, methyl cellulose, hydrogenated edible fats, gelatin, hydroxyalkylcelluloses, carboxymethylcellulose, aluminium stearate gel, hydrogenated edible fats) emulsifying agents (e.g. lecithin, sorbitan monooleate, or acacia), aqueous or non-aqueous vehicles (including edible oils, e.g. almond oil, fractionated coconut oil) oily esters (for example esters of glycerine, propylene glycol, polyethylene glycol or ethyl alcohol), glycerine, water or normal saline; preservatives (e.g. methyl or propyl p-hydroxybenzoate or sorbic acid) and conventional flavoring, preservative, sweetening or coloring agents. Diluents such as water, ethanol, propylene glycol, glycerin and combinations thereof may also be included.

Other suitable fillers, binders, disintegrants, lubricants and additional excipients are well known to a person skilled in the art.

The heat-stable F. prausnitzii compositions of the present invention may also be delivered as nutraceuticals, dietary supplements, nutritional supplements, or functional foods.

The dietary supplement of the present invention may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like. For example, the dietary supplement of the present invention may contain one or more of the following: asorbates (ascorbic acid, mineral ascorbate salts, rose hips, acerola, and the like), dehydroepiandosterone (DHEA), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), spirulina, zinc, and the like. Such optional ingredients may be either naturally occurring or concentrated forms. In preferred embodiments, the ingredients do not naturally occur in F. prausnitzii.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

In other embodiments, the present invention provides nutritional supplements (e.g., energy bars or meal replacement bars or beverages) comprising The heat-stable F. prausnitzii compositions of the present invention. In preferred embodiments, the nutritional supplements comprise an effective amount of the composition as described above. The nutritional supplement may serve as meal or snack replacement and generally provide nutrient calories. Preferably, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, wherein simple carbohydrates are mono- or disaccharides. The nutritional supplement contains, in one embodiment, combinations of sources of carbohydrate of three levels of chain length (simple, medium and complex; e.g., sucrose, maltodextrins, and uncooked cornstarch).

In still further embodiments, the present invention provides food products, prepared food products, or foodstuffs (i.e., functional foods) comprising The heat-stable F. prausnitzii compositions of the present invention. In preferred embodiments, the foods comprise an effective amount of the composition as described above. For example, in some embodiments, beverages and solid or semi-solid foods comprising the heat-stable F. prausnitzii compositions of the present invention are provided. These forms can include, but are not limited to, beverages (e.g., soft drinks, milk and other dairy drinks, and diet drinks), baked goods, puddings, dairy products, confections, snack foods, or frozen confections or novelties (e.g., ice cream, milk shakes), prepared frozen meals, candy, snack products (e.g., chips), soups, spreads, sauces, salad dressings, prepared meat products, cheese, yogurt and any other fat or oil containing foods, and food ingredients (e.g., wheat flour).

### EXPERIMENTAL

The following examples are provided in order to demonstrate and further illustrate certain preferred embodiments and aspects of the present invention and are not to be construed as limiting the scope thereof.

### Example 1

### Materials

1. Faecalibacteria prausnitzii supernatant. The cell free F. prausnitzii supernatant was obtained from 5-day cultures of F. prausnitzii in an anaerobic chamber. After centrifugation and filter sterilization, the supernatant was stored at −20° C. Five strains were cultured: Swine (266,267), Bovine (24,30), and Human (DSM). All cultures were in VTR2-RF medium (VTR2 medium with ruminal fluid).
2. Sephadex G25. The Sephadex G25 beads was purchased from Sigma Aldrich. The dry beads were soaked in M9 (33.7 mM Na₂HPO₄, 22 mM KH₂PO₄, 8.55 mM NaCl) buffer for 24 hours before use.

### Methods

1. Heat inactivation. Samples in microcentrifuge tubes were boiled for 3 min. The heated samples were kept on ice and tested for growth inhibition.
2. Sample Dialysis. Dialysis tubing with molecular weight cut-off of 6 KD (Spectrum Laboratory, Inc., CA) was used to dialyze the fresh medium (VTR2RF), and F. prausnitzii supernatant against PBS. Fresh PBS was used every 24 h. The dialysis was run for 48 hours at 4° C.
3. Size exclusion chromatography. Sephadex G25 column (5 mL) was used to fractionate F. prausnitzii supernatant. Briefly, 1 mL of cell free supernatant was transferred on the Sephadex G25 column pre-equilibrated with 10 mL of M9 medium (no N or C-source). The column was washed stepwise with 2 mLof M9 salt buffer for 9 times. All fractions were collected in 5 mL sterile tubes labelled with flowthrough, and F1 through F9. All fractions were tested along with the un-fractionated control samples in a standard growth curve assay (Bioscreen System, USA). The fractions with inhibitory activity were considered active fractions. These fractions were pooled together and concentrated with Amicon Ultra-15 Centrifigular Filter (Millipore, Ultrcel-3K). Proximately 10 mL of the pooled active fractions were centrifuged in the concentrator for 10 min at 4000×g at 4° C. This step was repeated until the volume reduced to about 1 mL. The flow through was saved for growth inhibition assays.

### Results

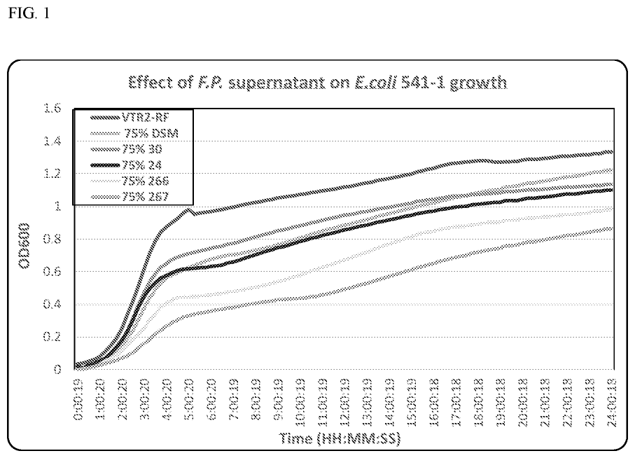

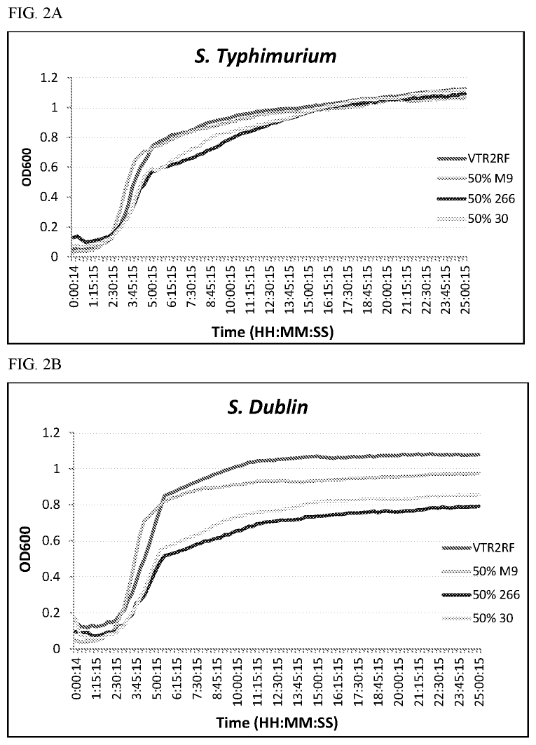

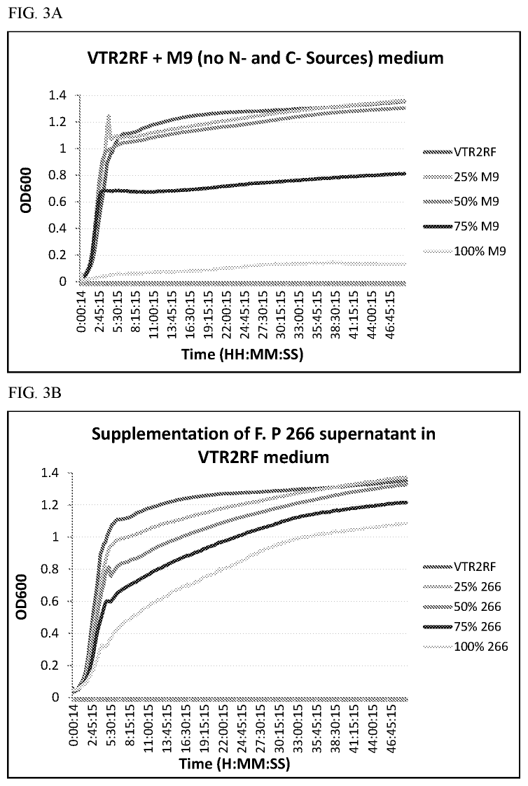

FIG. 1 provides a graph demonstrating the effect of F. prausnitzii supernatant on 30 E. coli 541-1 growth. Swine strains (266 and 267) are the strongest inhibitors (>56%). Similar inhibitory effects were found for AIEC E. coli LF82 and 524-2. An inhibitory effect was also observed for Salmonella spp. See FIGS. 2A and 2B. These data show that F. prausnitzii culture supernatants have an antibacterial effect against E. coli and Salmonella spp. The reduced growth is not due to nutrient depletion. Fresh F. prausnitzii growth medium (VTR2RF) was used either alone, or mixed with either M9 medium (no N- or C-sources) (FIG. 3A) or F. prausnitzii 266 supernatant (FIG. 3B) at given percentage. See FIGS. 3A and 3B.

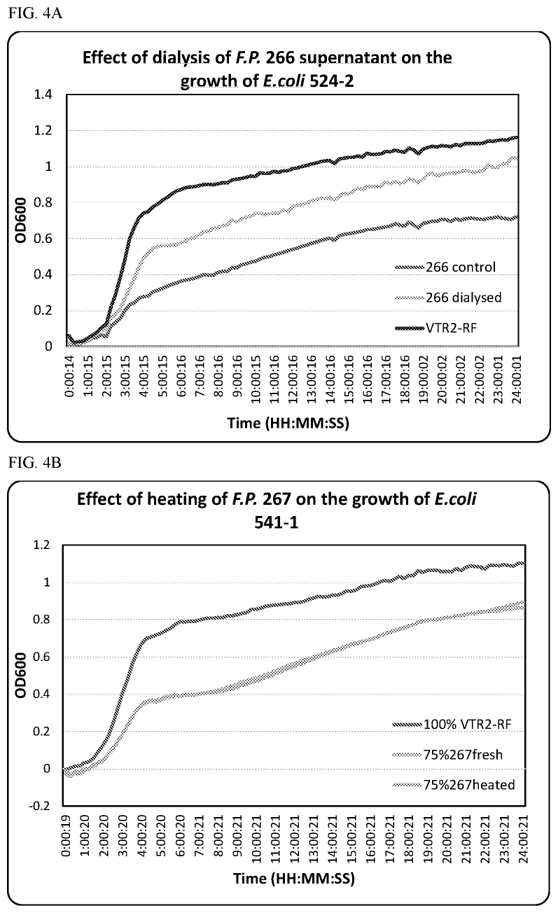

FIGS. 4A and 4B provide data demonstrating the effect of dialysis (6-8 kDa) and boiling on growth inhibition. Briefly, 10 ml of F. prausnitzii 266 supernatant was dialyzed against 1 L PBS for 24 hours at 4° C. on a stirring plate. The sample was recovered and filter sterilized before testing. The inhibitory effect is abrogated by dialysis (6-8K DaMW cutoff) indicating that the inhibitory effector in the supernatant is dialyzable. Heat treatment does not destroy the inhibitory effect of the neat (non-dialyzed) supernatant.

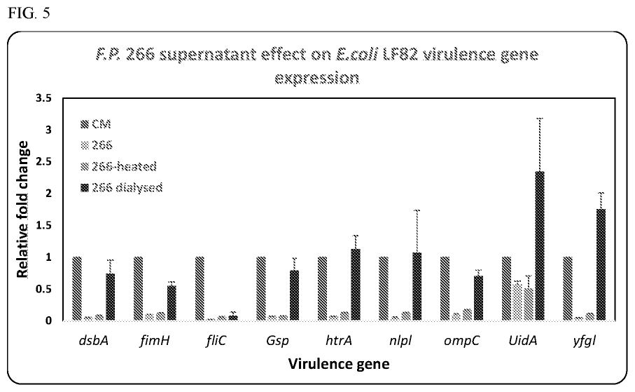

FIG. 5 provides a graph demonstrating the effect of F. prausnitzii 266 supernatant on E. coli LF82 virulence. The data shows downregulation of AIEC virulence genes by F. prau secreted products, demonstrating a bacterial taming effect. Heating (100° C. for 3 min) does not alter reduction in gene expression. Dialysis (MW cutoff=6-8K) largely restores gene expression. This suggests a mix of different molecular weight heat stable secreted products. Additional data (not shown) demonstrated that F. prausnitzii secreted products from bovine strain 30 and human DSM activated the uidA stress response in AIEC. The active constituent was non-dialysable(6-8 kDa) and heat-inactivated.

Taken together, these data show that F. prausnitzii secreted products inhibit the growth of AIEC associated with IBD, as well as Klebsiella pneumonia and Salmonella spp. F. prausnitzii secreted products also suppress virulence gene expression of Crohn's associated AIEC resulting in bacterial taming. The active constituents are heat-stable and a mix of secreted products <6-8 kDa and >6-8 kDa. F. prausnitzii swine strains have the most consistent and strong growth inhibition. A subset of F. prausnitzii secrete a heat-labile product >8 kDa that induces an E. coli uidA stress response.

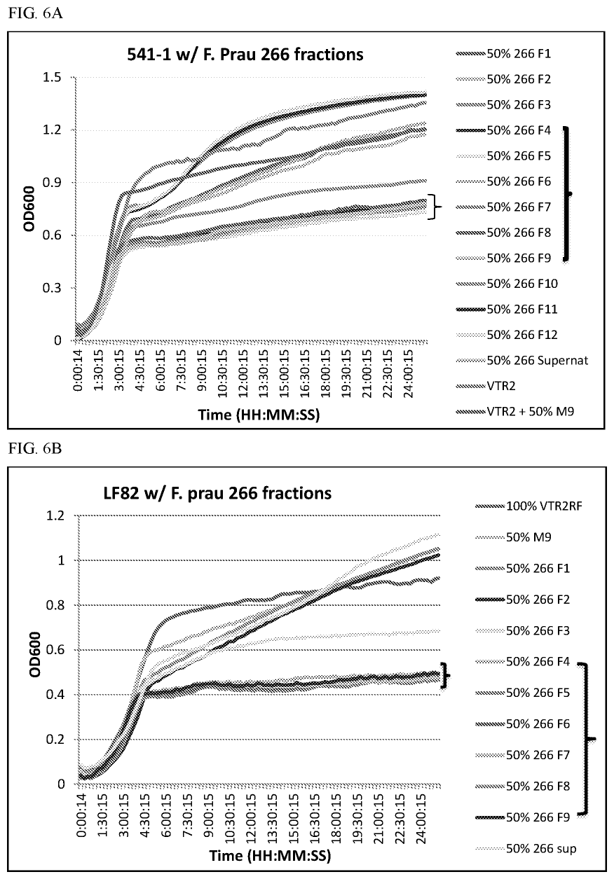

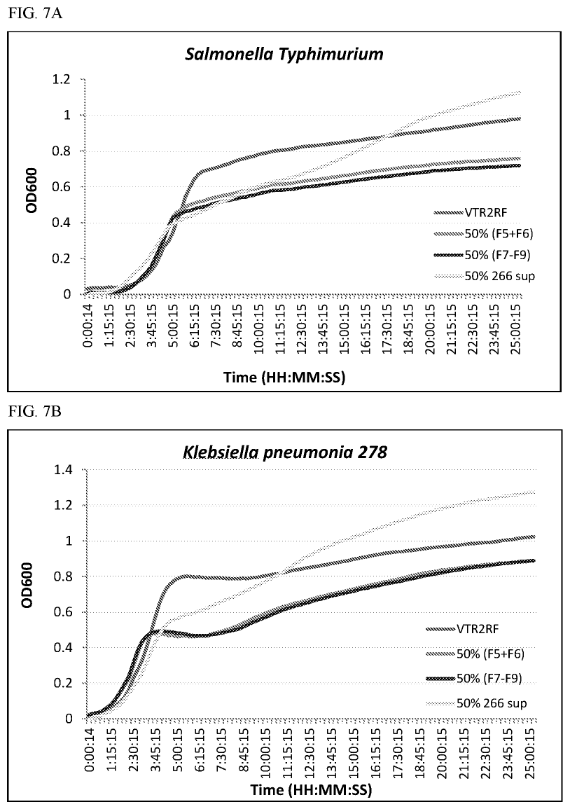

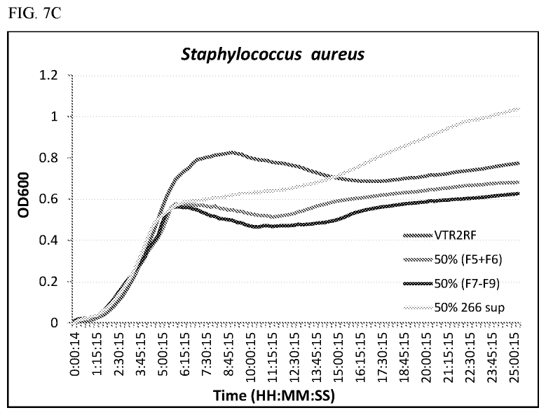

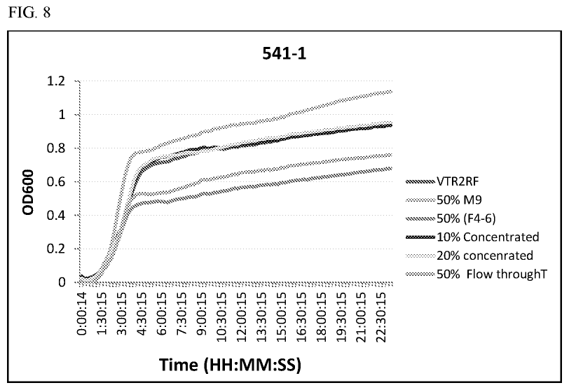

We conducted further experiments to characterize the antibacterial and bacterial taming products. FIGS. 6A and 6B provides the results from Sephedex G25 filtration of the supernatants. Sephadex gel filtration (G25: 7 kDA cutoff) of F. prau culture supernatant reveals that fractions 3 and fractions 4 through nine have strong antibacterial activity against AIEC 541-1 and LF82. FIGS. 7A, 7B and 7C demonstrate that heat stable filterable secreted products of F. prausnitzii 266 inhibit the growth of Salmonella (7A), Klebsiella (7B) and Staphylococcus spp. (7C). FIG. 8 is a graph of data demonstrating that the predominant antibacterial effect of F.prau secreted products is mediated by constituents <3 Kda. Briefly, pooled Sephadex G25 fractions (F4-F6, 6 ml) were concentrated with a 3Kda MW cutoff Centricon filter. The retained fraction and the flow through were tested against E. coli AIEC 541-1. The flow through suppressed growth of AIEC 541-1. Similar results were obtained for pooled Sephadex G25 fractions F7-F9 (6 ml).

In summary, we have discovered that secreted products from F. prausnitzii have previously unrecognized antibacterial activity against E. coli, Klebsiella spp. a Salmonella spp. and Staphylococcus spp. We have discovered that secreted products from F. prausnitzii have previously unrecognized bacterial taming ability against E. coli associated with IBD/Crohn's disease. We have determined that the “antibacterial” and “bacterial taming” activity against E. coli is heat stable and filterable. Sephadex G25 Gel filtration indicates that antibacterial activity is concentrated in fractions 3 and 4-9 and is comprised of at least two distinct constituents.

### Example 2

This example describes additional experiments on F. prausnitzii supernatants, their stability, their ability to inhibit growth of pathogenic organisms, and their ability to tame the virulence of pathogenic bacteria.

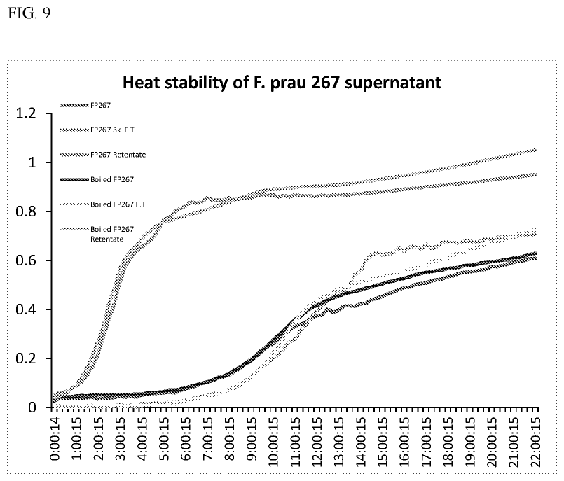

Heat stability of F. prausnitzii 267 supernatant antibacterial activity. F. prausnitzii 267 supernatant was collected after 24 h growth. Part of the cell free supernatant was filtered through Amicon 3K filter. Aliquots of the supernatant, 3 k filtrate, and 3 K retentate were boiled for 5 min, then kept on ice until tested with E. coli 541-1 in growth experiments. The data is presented in FIG. 9 which shows that antibacterial activity of F.prau secreted products is heat stable and contained within 3 k flowthrough.

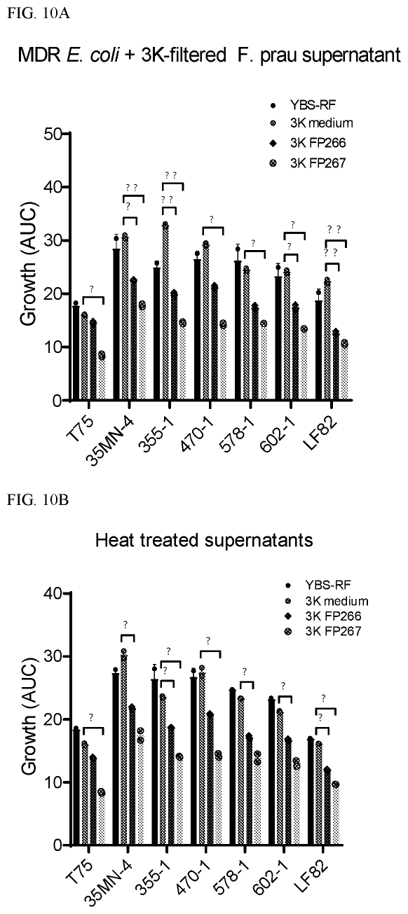

Filtered and heat treated F. prausnitzii secreted products inhibit growth of MDR E. coli. The supernatants of FP266 and FP267 were filtered through 3K Amicon filters. The flowthrough (75%) was added to the growth media (YBS-RF). The 3K flowthrough of YBS-RF was used as Control. The 3K filtrates of FP266 and FP267 supernatants, and the control medium were boiled for 10 min before testing. Growth determined by Biolog analyzer is reported as area under the curve (AUC). The data is presented in FIGS. 10A and 10B, which show that filtered and heat treated F. prausnitzii 3K filtrate inhibits the growth of multiple drug resistant (MDR) E. coli strains.

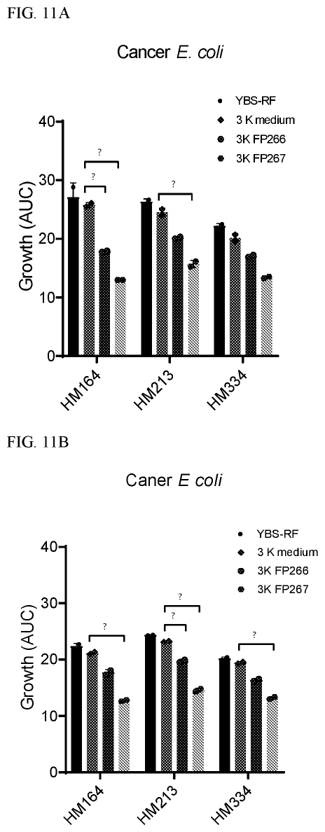

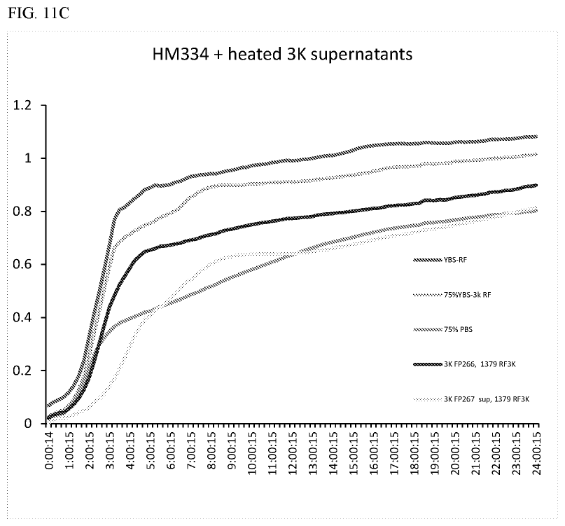

Filtered and heat treated F. prausnitzii secreted products inhibit growth of E. coli strains associated with colon cancer. The supernatants of FP266 and FP267 were filtered through 3K Amicon filters. The flowthrough (75%) was added to the growth media (YBS-RF). The 3K flowthrough of YBS-RF was used as Control. The 3K filtrates of FP266 and FP267 supernatants, and the control medium were boiled for 10 min before testing. Growth determined by Biolog analyzer is reported as area under the curve (AUC). The data is presented in FIGS. 11A, 11B, and 11C which show that filtered and heat treated F. prausnitzii 3K filtrate inhibits the growth of E. coli strains that are associated with the development of colon cancer (e.g., E. coli HM164, HM213 and HM334).

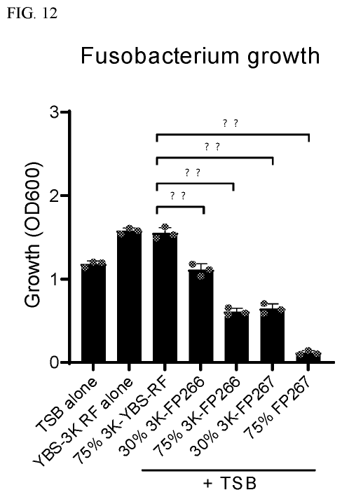

F. prausnitzii (266,267) secreted products inhibit growth of Fusobacterium nucleatum which has been associated with intestinal cancer in people. F. nucleatum was grow in TSB medium (Tryptic soy broth, fusobacterium growth medium) for 2 days. This culture was diluted (1 to 50) into media containing 3K supernatant of FP266 or FP267 as indicated. The optical density of each sample was measured at 48 h post inoculation. The 3K medium (3K YBS-RF) was used as control. The data is presented in FIG. 12, which shows that the 3K filtrate inhibits growth of F. nucleatum.

Human F. prausnitzii DSM17677 secreted products inhibit growth of AIEC, Salmonella typhi, Listeria and S. aureus.

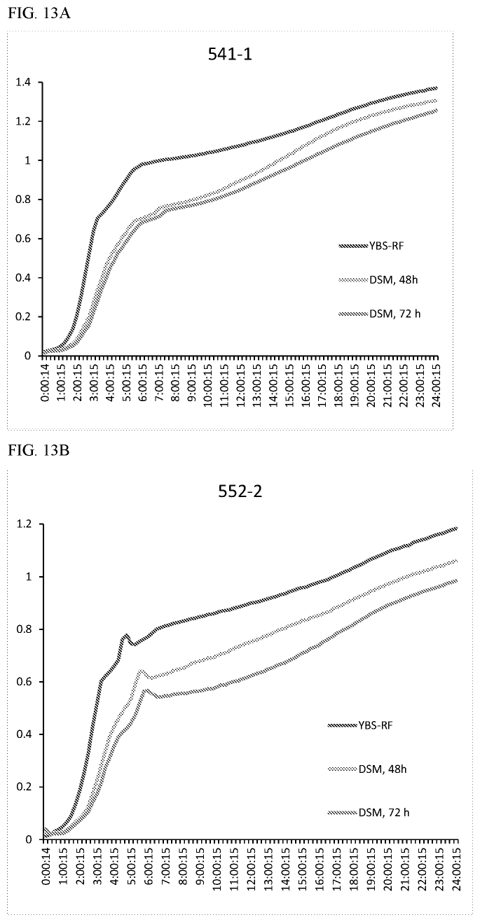

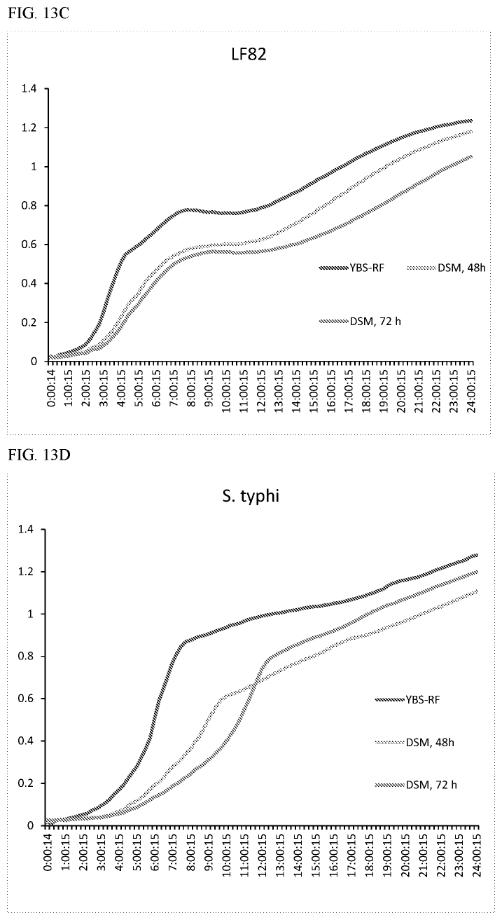

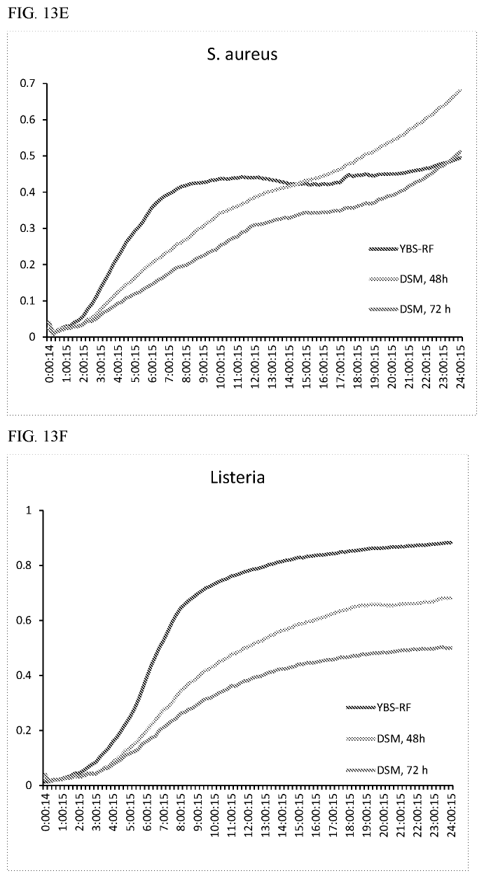

The ability of the secreted products in DSM17677 supernatant to inhibit the growth of various types of pathogenic organisms was examined. The data is presented in FIGS. 13A-F, which show that the DSM17677 filtrate inhibits the growth of AIEC (Adherent Invasive E. coli) E. coli strains (e.g., strains 541-1, 552-2 and LF82), Salmonella typhi, Listeria and S. aureus.

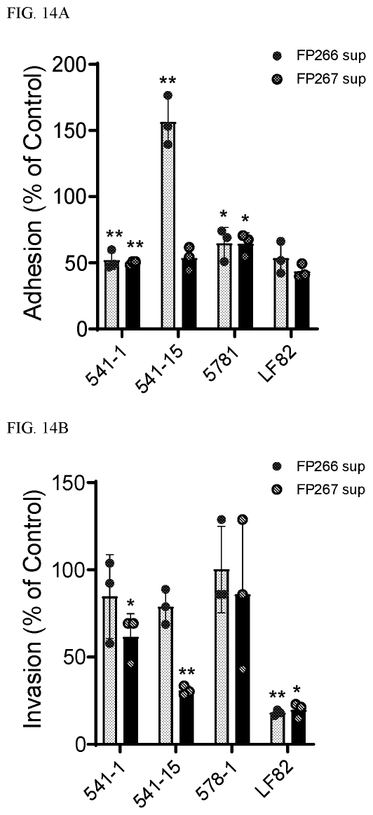

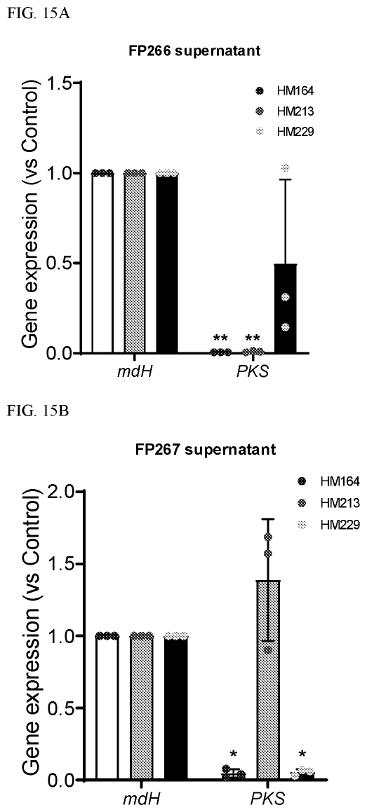

F. prausnitzii secreted products can reduce the ability of AIEC to adhere to and invade intestinal epithelial cells (Caco2) in vitro. E. coli was grown in LB overnight. Caco2/J774 cells were infected with inoculum containing either 50% of 3K YBS-RF (Control) or F. prau supernatant (3K filtered). The data is presented in FIGS. 14A and 14B. Adhesion and Invasion by AIEC type strain LF82 was reduced by F. prau 266 and 267.

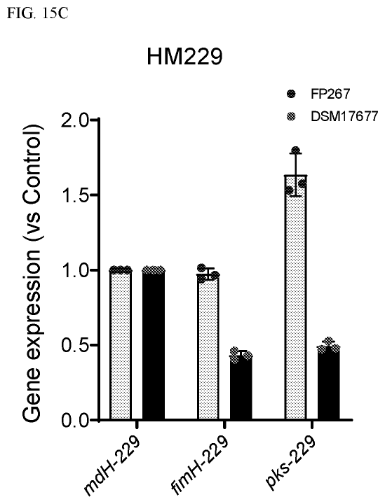

F. prausnitzii secreted products inhibit transcription of the pks gene in cancer-associated E. coli. E. coli was grown in either 75% YBS-3K RF (Control) or F. prausnitzii supernatant (3K-RF) until OD600 reached to about 1. Two volumes of RNAProtect reagent were added to preserve RNA integrity. Total RNA was isolated, and qRT-PCR for house keeper mdh and cancer associated pks (polyketide synthase was performed. The data is presented in FIGS. 15A, 15B and 15C. F. prausnitzii supernatants from 266, 267 and DSM (human) downregulated expression of the cancer associated PKS gene in E. coli strains associated with CRC.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A composition comprising a heat-stable secreted compound(s) from F. prausnitzii, the compound(s) characterized in having a molecular weight of less than 10 kDa.

2. A composition comprising a F. prausnitzii supernatant fraction characterized in comprising heat-stable compounds having a molecular weight of less than 10 kDa and being substantially free of compounds of greater than 10 kDa in weight.

3. A composition comprising heat-stable secreted compound(s) from F. prausnitzii.

4. The composition of any one of claims 1 to 3, wherein the compound(s) are characterized in having a molecular weight of less than 8 kDa.

5. The composition of any one of claims 1 to 3, wherein the compound(s) are characterized in having a molecular weight of less than 6 kDa.

6. The composition of any one of claims 1 to 3, wherein the compound(s) are characterized in having a molecular weight of less than 5 kDa.

7. The composition of any one of claims 1 to 3, wherein the compound(s) are characterized in having a molecular weight of less than 3 kDa.

8. The composition of any one of claims 1 to 7, wherein the composition comprises heat-stable secreted compound(s) from a single F. prausnitzii strain.

9. The composition of any one of claims 1 to 7, wherein the composition comprises heat-stable secreted compound(s) from a two or more F. prausnitzii strains.

10. The composition of any one of claims 1 to 7, wherein the composition comprises heat-stable secreted compound(s) from one to five F. prausnitzii strains.

11. The composition of any one of claims 1 to 7, wherein the composition comprises heat-stable secreted compound(s) from one to three F. prausnitzii strains.

12. The composition of any one of claims 1 to 11, wherein the heat-stable compounds are obtained by culturing F. prausnitzii to provide a culture supernatant and heating the culture supernatant under conditions sufficient to denature proteins in the supernatant.

13. The composition of claim 12, wherein the conditions sufficient to denature proteins in the supernatant comprise heating the supernatant to 100 degrees Celsius for 3 minutes.

14. The composition of any one of claims 12 to 13, wherein the culture supernatant is filtered or dialyzed to provide a fraction comprising molecules of less than 10 kDa.

15. The composition of any one of claims 1 to 14, further comprising a pharmaceutically acceptable excipient or carrier.

16. The composition of claim 15, wherein the pharmaceutically acceptable excipient or carrier is not naturally associated with the F. prausnitzii heat-stable secreted compound(s).

17. The composition of any one of claims 1 to 16, wherein the composition is formulated as a powder, bolus, gel, liquid drench, capsule, tablet, emulsion, syrup, gummi or paste.

18. The composition of any one of claims 1 to 17, wherein the composition comprises at least a second active agent.

19. The composition of claim 18, wherein the at least a second active agent is selected from the group consisting of a prebiotic agent, a probiotic agent, and anti-inflammatory agent and an anti-bacterial agent and combinations thereof.

20. A method of inhibiting or reducing bacterial growth in a subject in need thereof comprising administering a composition of any one of claims 1 to 19 to the subject.

21. Method of claim 20, wherein the bacteria is selected from the group consisting of E. coli, Klebsiella, Staphylococcus aureus, Salmonella spp., Listeria spp., and F. nucleatum.

22. Method of claim 21, wherein the E. coli is a Multiple Drug Resistant (MDR) E. coli strain.

23. Method of claim 21, wherein the E. coli is an adherent invasive E. coli (AIEC) strain.

24. Method of claim 23, wherein the adherence and/or invasiveness of the AIEC strain is reduced or inhibited.

25. Method of claim 21, wherein the E. coli is a cancer-associated E. coli strain.

26. Method of claim 25, wherein expression of the polyketide synthase gene (pks) is reduced or inhibited.

27. Composition of any one of claims 1 to 19 for use in inhibiting bacterial growth, treating a bacterial infection, or preventing a bacterial infection in a subject in need thereof.

28. Composition of claim 27, wherein the bacteria is selected from the group consisting of E. coli, Klebsiella, Staphylococcus aureus, Salmonella spp., Listeria and F. nucleatum.

29. Composition of claim 27, wherein the E. coli is a Multiple Drug Resistant (MDR) E. coli strain.

30. Composition of claim 27, wherein the E. coli is an adherent invasive E. coli (AIEC) strain.

31. Composition of claim 30, wherein the adherence and/or invasiveness of the AIEC strain is reduced or inhibited.

32. Composition of claim 28, wherein the E. coli is a cancer-associated E. coli strain.

33. Composition of claim 32, wherein expression of one or more polyketide synthase (pks) island genes is reduced or inhibited.

34. A method of reducing the virulence of a bacteria in a subject in need thereof comprising administering a composition of any one of claims 1 to 19 to the subject.

35. The method of claim 34, wherein the bacteria is E. coli.

36. Method of claim 35, wherein the E. coli is a Multiple Drug Resistant (MDR) E. coli strain.

37. Method of claim 35, wherein the E. coli is an adherent invasive E. coli (AIEC) strain.

38. Method of claim 37, wherein the adherence and/or invasiveness of the AIEC strain is reduced or inhibited.

39. Method of claim 35, wherein the E. coli is a cancer-associated E. coli strain.

40. Composition of any one of claims 1 to 19 for use in inhibiting, treating or preventing virulence of a bacteria in a subject in need thereof.

41. Composition of claim 40, wherein the bacteria is E. coli.

42. Composition of claim 41, wherein the E. coli is a Multiple Drug Resistant (MDR) E. coli strain.

43. Composition of claim 41, wherein the E. coli is an adherent invasive E. coli (AIEC) strain.

44. Composition of claim 43, wherein the adherence and/or invasiveness of the AIEC strain is reduced or inhibited.

45. Composition of claim 41, wherein the E. coli is a cancer-associated E. coli strain.

46. A method of treating, inhibiting or preventing acute gastroenteritis, traveler's diarrhea, irritable bowel syndrome, Crohn's disease or ulcerative colitis in a subject in need thereof comprising administering a composition of any one of claims 1 to 19 to the subject.

47. Use of a composition of any one of claims 1 to 19 to treat, prevent or inhibit acute gastroenteritis, traveler's diarrhea, irritable bowel syndrome, Crohn's disease or ulcerative colitis in a subject in need thereof.

48. A method of inhibiting expression of the polyketide synthase gene (pks) gene by cancer-associated E. coli strains in a subject in need thereof comprising administering to the subject a composition according to any one of claims 1 to 19.

49. Use of a composition of any one of claims 1 to 19 to prevent or inhibit expression of the polyketide synthase gene (pks) gene by cancer-associated E. coli strains in a subject in need thereof.

50. A method of preventing or inhibiting adherence and/or invasiveness by AIEC E. coli strains in a subject in need thereof comprising administering to the subject a composition according to any one of claims 1 to 19.

51. Use of a composition of any one of claims 1 to 19 to prevent or inhibit adherence and/or invasiveness by AIEC E. coli strains in a subject in need thereof.

52. A food product comprising a composition of any one of claims 1 to 19 in combination with one or more food ingredients selected from the group consisting of a fat, a carbohydrate and a protein.

53. The food product of claim 52, wherein the one or more food ingredients does not naturally occur in F. prausnitzii.

54. The food product of either of claims 52 and 53, wherein the one or more food ingredient is obtained from a source other than F. prausnitzii.

55. The food product of any one of claims 52 to 54, wherein the food product is a solid food.

56. The food product of any one of claims 52 to 54, wherein the food product is a semi-solid food.

57. The food product of any one of claims 52 to 54, wherein the food product is a beverage.

58. The food product of any one of claims 52 to 54, wherein the food product is a baked good.

59. The food product of any one of claims 52 to 54, wherein the food product is a food bar.
